(19) United States
(12) Patent Application Publication
(10) Pub. No.: US 2023/0414581 A1
(43) Pub. Date: Dec. 28, 2023

(54) SARM1 ENZYME ACTIVITY INHIBITOR AND USE THEREOF IN NEURODEGENERATIVE DISEASES

(71) Applicant: ARTIVILA BIOPHARMA, Wuxi, Jiangsu (CN)

(72) Inventors: Deqiang NIU, Wuxi, Jiangsu (CN); Zhendong ZHU, Wuxi, Jiangsu (CN); Yongjuan ZHAO, Wuxi, Jiangsu (CN); Wanhua LI, Wuxi, Jiangsu (CN)

(73) Assignee: ARTIVILA BIOPHARMA, Wuxi, Jiangsu (CN)

(21) Appl. No.: 18/036,673

(22) PCT Filed: Nov. 9, 2021
(86) PCT No.: PCT/CN2021/129518

§ 371 Date: May 12, 2023

(30) Foreign Application Priority Data
Nov. 12, 2020 (CN) 202011264827.5

(51) Int. Cl. A61K 31/433

(52) U.S. Cl. CPC A61K 31/433

(57) ABSTRACT

Provided in the present disclosure is the use of an SARM1 enzyme activity inhibitor in the treatment of neurodegenerative diseases or neurological diseases or conditions, and in particular, provided in the present invention are a compound represented by formula (a) as an SARM1 enzyme activity inhibitor, and a pharmaceutical composition thereof.

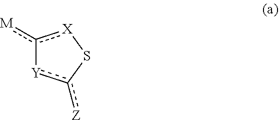

### CROSS REFERENCE TO RELATED APPLICATIONS

This application claims the priority of Chinese Patent Application No. 202011264827.5, filed with the China National Intellectual Property Administration on Nov. 12, 2020, and titled with “SARM1 ENZYME ACTIVITY INHIBITOR AND USE THEREOF IN NEURODEGENERATIVE DISEASES”, which is hereby incorporated by reference in its entirety.

### FIELD

The present application relates to compounds that can be used to inhibit SARM1 enzyme activity, and/or use of the compounds in the treatment and/or prevention of neurodegenerative or neurological diseases or conditions related to the SARM1 enzyme activity.

### BACKGROUND

Neurodegenerative diseases are a class of diseases that can be seriously harmful for humans, which can cause devastating damage, such as progressive diseases of neuronal cell death. Primary neurological degenerative diseases that have been known include central nervous system diseases such as Alzheimer's disease, Parkinson's disease, amyotrophic lateral sclerosis (ALS), and Huntington's disease and peripheral neurological diseases such as diabetes. Most of these diseases are related to aging, and in fact the onset of these diseases increases with age, although there are also cases of middle-aged and even younger people.

As a result of studies on brain structure and function, the roles of neurotransmitters and neurotrophic factors have been gradually elucidated, but many local causes of neurological degeneration remain unclear. Only the relationship between Parkinson's disease and a special neurotransmitter, i.e., dopamine, has been clarified. L-dopa, the precursor of dopamine, has been used as a drug for alleviating neurological symptoms and restoring neurological function. However, L-dopa cannot inhibit the development of neurological degeneration, and gradually loses effect with the development of the disease, i.e., the degeneration and defect of dopamine-based nerve cells. Similarly, Alzheimer's disease is also caused by the degeneration and defect of various nerve cells such as acetylcholine-based nerve cells and monoamine-based nerve cells. As a drug for treating this disease, cholinesterase inhibitors have been on the market or being developed. However, L-dopa for the treatment of Parkinson's disease is still limited to the treatment of symptoms to temporarily alleviate neurological symptoms.

Therefore, for neurodegenerative diseases, effective therapeutic drugs are particularly lacking to date.

Studies have shown that axon damage occurs in a variety of nervous system diseases such as neurodegenerative diseases and accidental injuries. Axonal degeneration can cause structural necrosis and dysfunction of the peripheral nervous system, eventually leading to acquired or inherited central nervous system degeneration.

Although there is no very effective pharmacological method to accurately assess the weight function of the morbidity caused by axonal degeneration, it has been found in histopathological studies that at the early stage of various neuropathies such as Alzheimer's disease, Parkinson's disease, multiple sclerosis, amyotrophic lateral sclerosis and peripheral neuropathy, significant axonal damage and degradation is observed, indicating that axonal degeneration plays an important role in the occurrence and development of neuropathy (Fischer et al., Neuro-degenerative Diseases, 2007, 4:431-442). Therefore, maintaining the structural and functional integrity of neurons by attenuating or even blocking axonal degeneration may be a therapeutic strategy for a variety of neurological diseases.

In the absence of effective therapeutic drugs for neurodegenerative diseases, there is an urgent need to research and develop new compounds in the prior art, especially chemical small molecules, including compounds that have an effect on nerve axonal degeneration.

### SUMMARY

After long-term research, the present inventors unexpectedly discovered a class of compounds with significant inhibitory effect on SARM1 enzyme activity, and found that the compounds can attenuate axonal degeneration and can be used for treating or preventing neurodegenerative diseases and related conditions thereof.

SARM1 consists of three structural domains, which are an ARM (Armadillo/HEAT repeat) domain at nitrogen-terminal, two tandem SAM (Sterile alpha motif) domains, and a TIR (Toll/Interleukin Receptor) domain at carbon-terminal. Additionally, there is also a mitochondrial localization signal peptide at nitrogen terminal.

It is known that in wild-type neurons, axonal injury induces depletion of NAD⁺ and axonal degeneration. The knock-out of SARM1 inhibits axonal degeneration, and maintains NAD⁺ at a normal level, indicating that SARM1 promotes consumption of NAD⁺ and exacerbates axonal degeneration.

Milbrandt's group at the University of Washington School of Medicine, USA prepared the TIR domain of SARM1 (SARM1-TIR) and found that it has NAD⁺ hydrolase activity. Further, high-purity SARM1-TIR was obtained through restrict E. coli expression and purification experiments and cell-free expression systems, which finally demonstrated that SARM1-TIR can catalyze NAD⁺ to generate adenosine 5′-diphosphate ribose (ADPR) and cyclic adenosine 5′-diphosphate ribose (cADPR).

SARM1 is a multifunctional signaling enzyme that can catalyze various substrates such as NAD⁺, NADP⁺ and NA to generate signal molecules such as cADPR, ADPR and NAADP. In a variety of neurodegenerative diseases, SARM1 is activated, leading to the depletion of NAD⁺, further initiating a new cell death mechanism. The knock-out of SARM1 can inhibit axonal degeneration and disease progression. Therefore, SARM1 is considered a potential drug target for related neurological diseases, including TBI, AD, CIPN, ASL, etc.

In the present disclosure, a full-length SARM1 is prepared for NAD enzyme activity experiment, and used for screening to obtain the compound molecules with enzyme activity-inhibiting ability of the present invention.

Therefore, based on the above findings, in a first aspect, the present invention provides use of an SARM1 enzyme activity inhibitor in the preparation of a medicament for the treatment or prevention of a neurodegenerative disease or a neurological disease or condition.

In another aspect, the present invention provides use of an SARM1 enzyme activity inhibitor in the preparation of a medicament for the treatment or prevention of a disease or condition related to axonal degeneration.

In particular, the present invention provides a compound represented by formula (a), a pharmaceutically acceptable salt or a prodrug thereof as an SARM1 enzyme activity inhibitor:

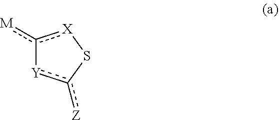

wherein,
X is selected from the group consisting of —NRₐ—, —N— and —S—,
M is selected from the group consisting of —NRₐR_{b}, —NRₐ, oxy (═O), —OR_{b} and —SR_{b},
Y is selected from the group consisting of —NRₐ—, —N═, ═CH— and ═CR_{c}—,
Z is selected from the group consisting of —NRₐR_{b}, —NRₐ, oxy (═O) and —OR_{b},
wherein Rₐ and R_{b} are each independently selected from the group consisting of hydrogen, C₁-C₁₀ alkyl, C₃-C₈ cycloalkyl, C₆-C₁₀ aryl, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl; wherein the C₁-C₁₀ alkyl, C₃-C₈ cycloalkyl, C₆-C₁₀ aryl, C₆-C₁₀ arylamino, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl are optionally substituted by 1, 2 or 3 substituents selected from the group consisting of halogen selected from the group consisting of fluorine, chlorine, bromine and iodine, nitro, cyano, C₁-C₃ alkyl, C₁-C₃ alkoxy, halogenated C₁-C₃ alkyl, halogenated C₁-C₃ alkylthio, and C₃-C₈ cycloalkyl C₁-C₃ alkyl;
wherein R_{c} is independently selected from the group consisting of hydrogen, —CN, —CO₂NHRₐ, —CO₂Rₐ, —NO₂, —CF₃ and Rₐ.

In a preferred aspect, the compound represented by formula (a) of the present invention is a compound represented by formula I:

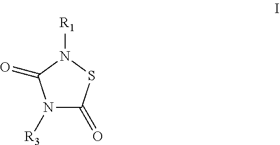

wherein, R₁ and R₃ are independently selected from the group consisting of: hydrogen, C₁-C₁₀ alkyl, C₃-C₈ cycloalkyl, C₆-C₁₀ aryl, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl; wherein the C₁-C₁₀ alkyl, C₃-C₈ cycloalkyl, C₆-C₁₀ aryl, C₆-C₁₀ arylamino, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl are optionally substituted by 1, 2 or 3 substituents selected from the group consisting of halogen selected from the group consisting of fluorine, chlorine, bromine and iodine, nitro, cyano, C₁-C₃ alkyl, C₁-C₃ alkoxy, halogenated C₁-C₃ alkyl, halogenated C₁-C₃ alkylthio, and C₃-C₈ cycloalkyl C₁-C₃ alkyl.

In another preferred aspect, the compound represented by formula (a) of the present invention is a compound represented by formula II-a or a compound represented by formula II-b:

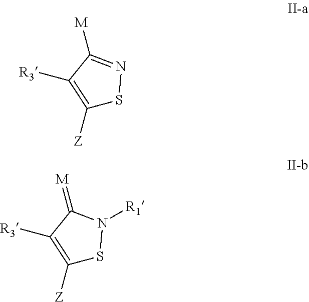

wherein,
in formula II-a, M is selected from the group consisting of —NRₐR_{b}, and in formula II-b, M is selected from the group consisting of oxygen, sulfur and —NRₐ;
Z is selected from the group consisting of —NRₐR_{b} and —OR_{b};
R₁′ is independently selected from the group consisting of Rₐ; R₃′ is independently selected from the group consisting of hydrogen, —CN, —CO₂NHRₐ, —CO₂Rₐ, —NO₂, —CF₃ and Rₐ;
wherein Rₐ and R_{b} are as defined above;
or, R₃′ and Z can be connected to form a 5-7 membered ring.

In yet another preferred aspect, the compound represented by formula (a) of the present invention is a compound represented by formula III:

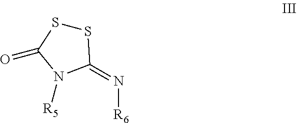

wherein R₅ and R₆ are independently selected from the group consisting of: hydrogen, C₁-C₁₀ alkyl, C₃-C₈ cycloalkyl, C₆-C₁₀ aryl, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl; wherein the C₁-C₁₀ alkyl, C₃-C₈ cycloalkyl, C₆-C₁₀ aryl, C₆-C₁₀ arylamino, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl are optionally substituted by 1, 2 or 3 substituents selected from the group consisting of halogen selected from the group consisting of fluorine, chlorine, bromine and iodine, nitro, cyano, C₁-C₃ alkyl, C₁-C₃ alkoxy, halogenated C₁-C₃ alkyl, halogenated C₁-C₃ alkylthio, and C₃-C₈ cycloalkyl C₁-C₃ alkyl.

In the compound represented by formula (a) of the present invention, preferably, Rₐ and R_{b} are independently selected from the group consisting of: C₁-C₃ alkyl; phenyl, benzyl and naphthyl, which are optionally substituted by substituents selected from the group consisting of methyl, isopropyl, trifluoromethyl, fluoro, chloro and nitro; cyclopropylmethyl; cyano and hydroxyl.

In the compounds represented by formula I, formula II-a, formula II-b and formula III of the present invention, preferably, R₁, R₃, R₁′, R₃′, R₅ and R₆ are each independently selected from the group consisting of: C₁-C₃ alkyl; phenyl, benzyl and naphthyl, which are optionally substituted by substituents selected from the group consisting of methyl, isopropyl, trifluoromethyl, fluoro, chloro and nitro; cyclopropylmethyl; cyano and hydroxyl.

In some more preferred embodiments, R₁ and R₃ in the compound represented by formula I are each independently selected from the group consisting of: methyl, benzyl, phenyl, naphthyl, p-methylphenyl, p-fluorophenyl, isopropylphenyl, trifluoromethylthiophenyl, nitro-, methyl- or chloro-substituted phenyl, cyclopropylmethyl, trifluoromethyl-substituted phenyl.

A particularly preferred compound of the present invention is selected from the group consisting of the following compounds, pharmaceutically acceptable salts and prodrugs thereof:

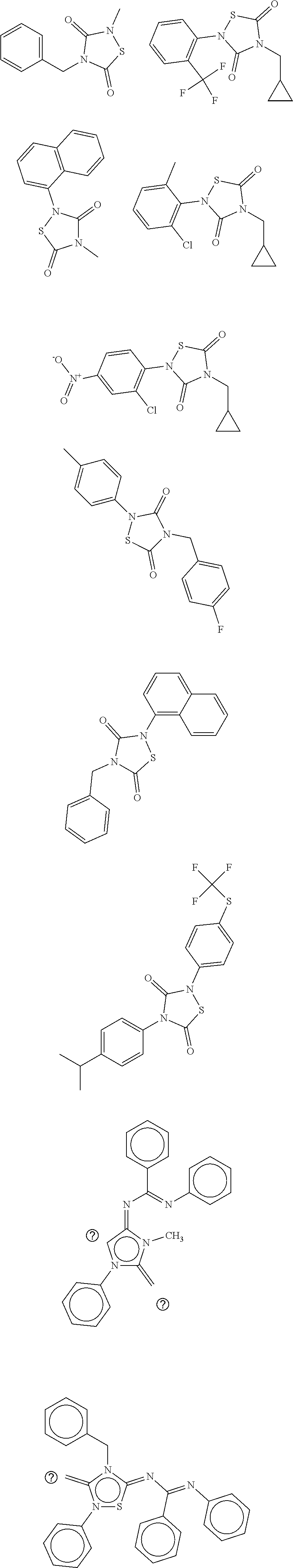

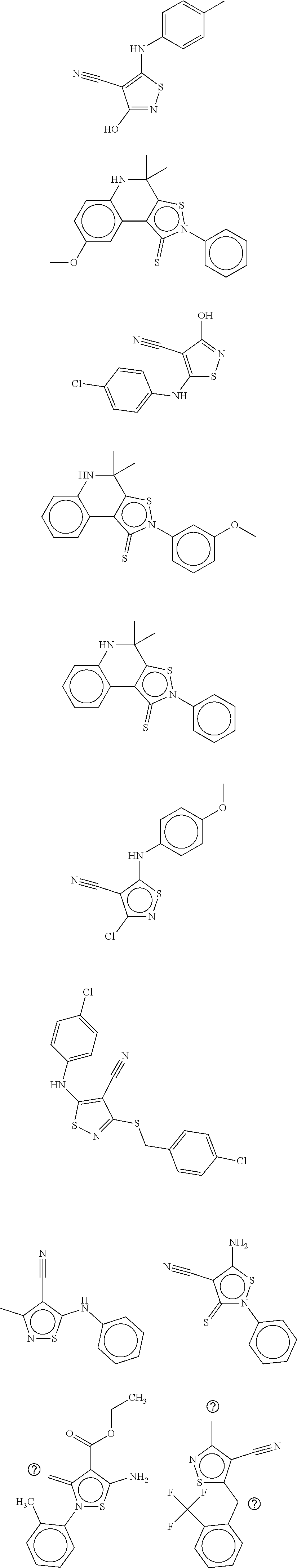

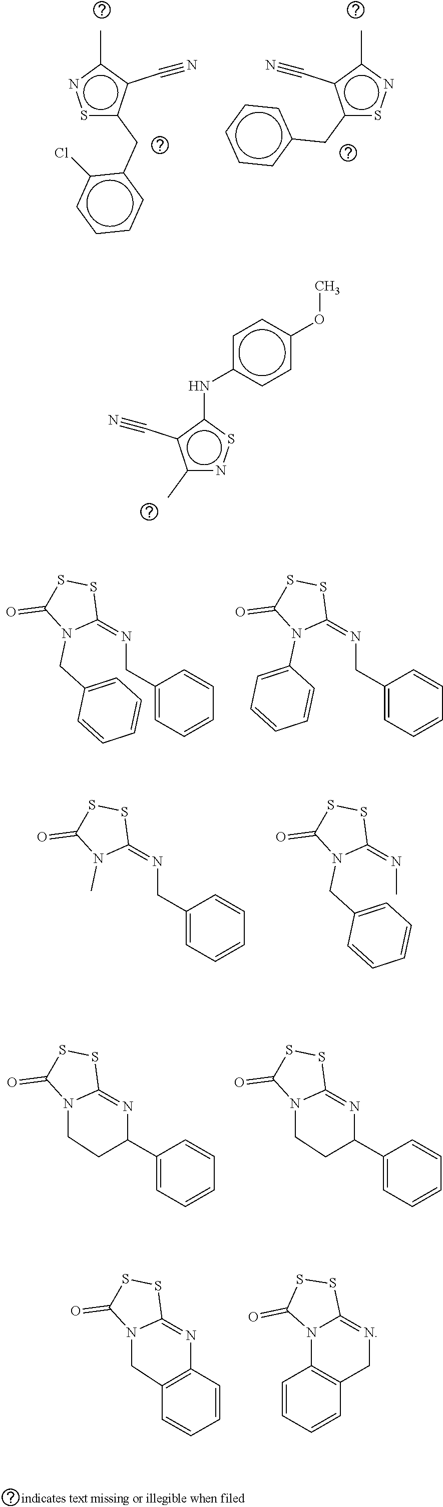

A more preferred compound of the present invention is selected from the group consisting of the following compounds, pharmaceutically acceptable salts and prodrugs thereof:

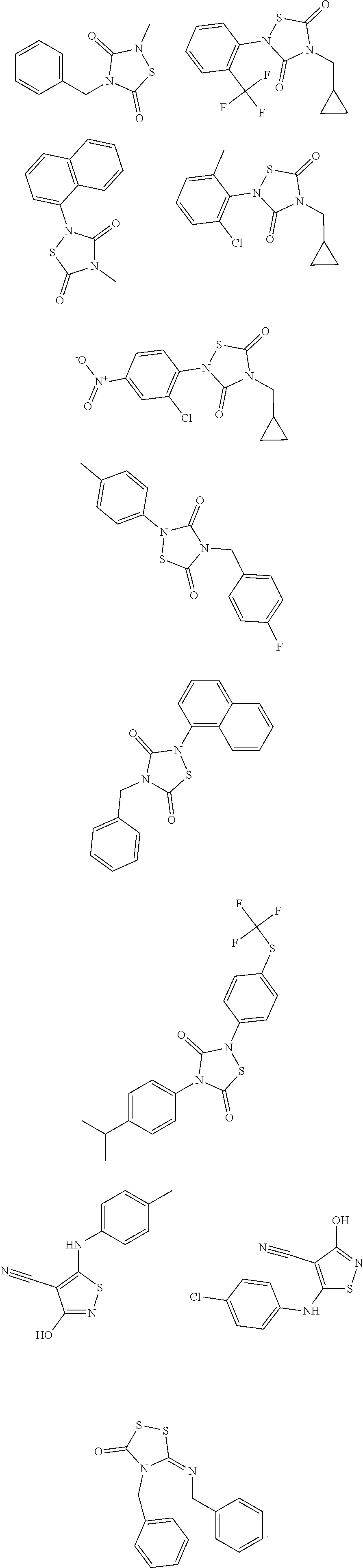

In another aspect, the present invention provides a compound represented by formula IV, a pharmaceutically acceptable salt or a prodrug thereof as an SARM1 enzyme activity inhibitor:

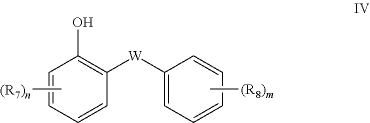

wherein,
W is selected from the group consisting of —CH₂—, —C(O)—, —O—, —S— and —NR₅—,
R₅ is selected from the group consisting of hydrogen, C₁-C₁₀ alkyl, C₃-C₈ cycloalkyl, C₆-C₁₀ aryl, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl; wherein the C₁-C₁₀ alkyl, C₃-C₈ cycloalkyl, C₆-C₁₀ aryl, C₆-C₁₀ arylamino, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl are optionally substituted by 1, 2 or 3 substituents selected from the group consisting of halogen selected from the group consisting of fluorine, chlorine, bromine and iodine, nitro, cyano, C₁-C₃ alkyl, C₁-C₃ alkoxy, halogenated C₁-C₃ alkyl, halogenated C₁-C₃ alkylthio, and C₃-C₈ cycloalkyl C₁-C₃ alkyl;
R₇ and R₈ are independently selected from the group consisting of hydroxyl, chlorine, bromine, C₁-C₁₀ alkyl and C₁-C₃ alkoxy;
m and n are independently selected from the group consisting of 0, 1, 2 and 3.

A preferred compound represented by formula IV is selected from the group consisting of the following compounds, pharmaceutically acceptable salts and prodrugs thereof:

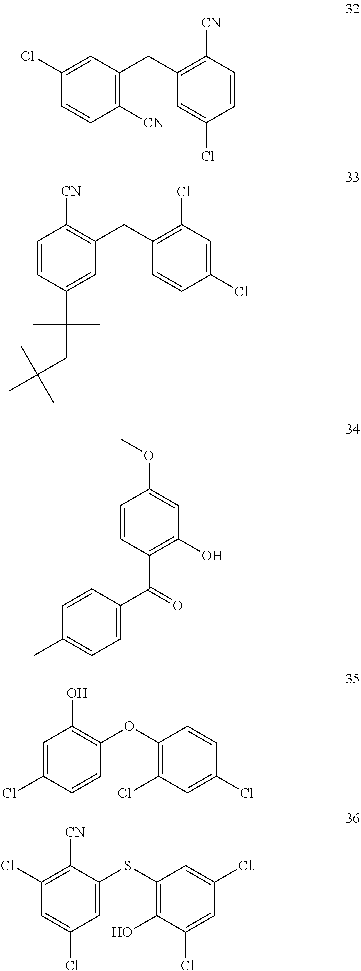

In another aspect, the present invention provides a compound selected from the following compounds, a pharmaceutically acceptable salt or a prodrug thereof as an SARM1 enzyme activity inhibitor:

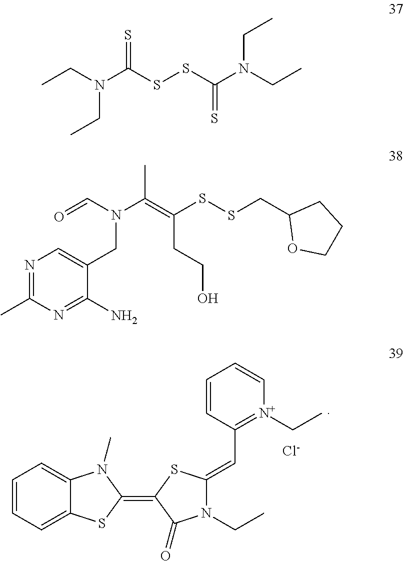

In another aspect, the present invention provides a compound represented by formula VI, a pharmaceutically acceptable salt or a prodrug thereof as an SARM1 enzyme activity inhibitor:

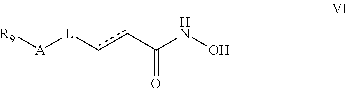

wherein,
L is selected from the group consisting of C₁-C₆ alkyl, C₆-C₁₀ aryl and C₆-C₁₀ heteroaryl, wherein the C₁-C₆ alkyl, C₆-C₁₀ aryl and C₆-C₁₀ heteroaryl are optionally substituted by 1 or 2 substituents selected from the group consisting of halogen selected from the group consisting of fluorine, chlorine and bromine, C₁-C₃ alkyl, C₁-C₃ alkoxy, and C₃-C₈ cycloalkyl;
A is selected from the group consisting of aminosulfonyl, aminoacyl and C₁-C₅ alkylamino;
R₉ is selected from the group consisting of C₆-C₁₀ aryl, C₆-C₁₀ heteroaryl, C₆-C₁₀ aryl C₁-C₃ alkyl and C₆-C₁₀ heteroaryl C₁-C₃ alkyl, wherein the C₆-C₁₀ aryl, C₆-C₁₀ heteroaryl, C₆-C₁₀ aryl C₁-C₃ alkyl and C₆-C₁₀ heteroaryl C₁-C₃ alkyl are optionally substituted by 1 or 2 substituents selected from the group consisting of halogen selected from the group consisting of fluorine, chlorine and bromine, C₁-C₃ alkyl, C₁-C₃ alkoxy, C₃-C₈ cycloalkyl, C₆-C₁₀ arylamino, and bis(C₆-C₁₀ aryl)amino.

In yet another aspect, the present invention provides a compound selected from the following compounds, a pharmaceutically acceptable salt or a prodrug thereof as an SARM1 enzyme activity inhibitor:

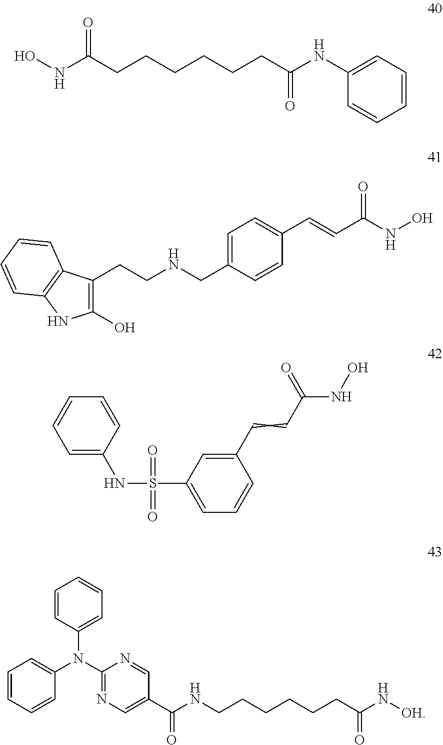 [SMILES: O=C(CCCCCCC(=O)Nc1ccccc1)NO ; O=C(/C=C/c1ccc(CNCCc2c(O)[nH]c3ccccc23)cc1)NO ; O=C(C=Cc1cccc(S(=O)(=O)Nc2ccccc2)c1)NO ; O=C(CCCCCCNC(=O)c1cnc(N(c2ccccc2)c2ccccc2)nc1)NO]

The present invention further provides a pharmaceutical composition, comprising the above compound of the present invention as an SARM1 enzyme activity inhibitor, and optionally a pharmaceutically acceptable carrier or excipient.

The present invention further provides a method for treating or preventing a neurodegenerative disease or a related neurological disease or condition, comprising administering the compound of the present invention as an SARM1 enzyme activity inhibitor to a subject in need thereof. In particular, the present invention provides a method for treating or preventing a disease or condition related to axonal degeneration, comprising administering the compound of the present invention as an SARM1 enzyme activity inhibitor to a subject in need thereof. More particularly, the present invention provides a method for inhibiting SARM1 enzyme activity, comprising administering the compound of the present invention to a subject in need thereof. More particularly, the present invention provides a method for inhibiting axonal degeneration, comprising administering the compound of the present invention to a subject in need thereof. The compound or composition of the present invention may be administered to a subject or patient in need thereof in an effective amount.

### DETAILED DESCRIPTION

### Terms

In the terms used in the present invention, “neurodegenerative disease” has the same meaning as “neurological degenerative disease”; and “axonal degeneration” has the same meaning as “axon degeneration”. Those skilled in the art can understand that the terms have commonly understood meanings.

Herein, when referring to a “compound” having a particular structural formula, a stereoisomer, a diastereomer, an enantiomer, a racemic mixture and an isotopic derivative thereof are generally also encompassed.

It is well known to those skilled in the art that, in addition to the salt of the compound, the solvate and hydrate of the compound are alternative forms of the compound, which can all be converted into the compound under certain conditions. Therefore, when referring to a compound herein, the solvate and hydrate thereof are generally included.

Similarly, when referring to a compound herein, the prodrug, metabolite and nitroxide thereof are also generally included.

The pharmaceutically acceptable salt of the present invention can be formed using, for example, the following inorganic or organic acids. “Pharmaceutically acceptable salt” refers to a salt which, within reasonable medical judgment, is suitable for use in contact with human and mammal tissues, without undue toxicity, irritation, allergic reaction, etc., and has a reasonable benefit/risk ratio. The salt can be prepared in situ during the final separation and purification of the compound of the present invention, or can be prepared by reacting a free base or acid with a suitable reagent. For example, a free base can be reacted with a suitable acid. In addition, when the compound of the present invention contains an acidic moiety, a suitable pharmaceutically acceptable salt thereof can include metal salt, such as alkali metal salt (such as sodium salt or potassium salt); and alkaline earth metal salt (such as calcium salt or magnesium salt). Examples of pharmaceutically acceptable non-toxic acid addition salt are salts formed by amino with inorganic acids (e.g., hydrochloric acid, hydrobromic acid, phosphoric acid, sulfuric acid, and perchloric acid) or organic acids (e.g., acetic acid, oxalic acid, maleic acid, tartaric acid, citric acid, succinic acid or malonic acid), or salts formed by using other methods known in the art such as ion exchange. Other pharmaceutically acceptable salts are selected from the group consisting of sodium alginate, ascorbate, besylate, adipate, camphorsulfonate, aspartate, benzoate, bisulfate, borate, butyrate, camphorate, citrate, dodecyl sulfate, ethanesulfonate, formate, fumarate, glucoheptonate, glycerophosphate, gluconate, heptanoate, hexanoate, hydroiodide, lactobionate, lactate, laurate, lauryl sulfate, malate, maleate, malonate, methanesulfonate, 2-naphthalenesulfonate, nicotinate, nitrate, oleate, oxalate, palmitate, pamoate, pectate, persulfate, 3-phenylpropionate, phosphate, picrate, pivalate, propionate, stearate, succinate, sulfate, tartrate, thiocyanate, p-toluenesulfonate, undecanoate, valerate, etc. Representative alkali or alkaline earth metal salts include salts of sodium, lithium, potassium, calcium, magnesium, and the like. Other pharmaceutically acceptable salts include, where appropriate, nontoxic ammonium salt, quaternary ammonium salt, and amine cation formed with a counterion, for example, halides, hydroxides, carboxylates, sulfates, phosphates, nitrates, lower alkyl sulfonates and aryl sulfonates.

The pharmaceutically acceptable salt of the present invention can be prepared by conventional methods, for example, by dissolving the compound of the present invention in a water-miscible organic solvent (such as methanol, ethanol, acetone and acetonitrile), adding an excess amount of an aqueous solution of organic acid or inorganic acid, so that precipitating a salt from the resulting mixture, removing the solvent and remaining free acid therefrom, and separating the precipitated salt.

“Solvate” as used herein refers to a physical association of the compound of the present invention with one or more solvent molecules, whether the solvent is organic or inorganic. This physical association includes hydrogen bonding. In some cases, for example, when one or more solvent molecules are incorporated into a crystal lattice of the crystalline solid, the solvate can be isolated. Solvent molecules in a solvate can exist in a regular and/or a disordered arrangement. A solvate can contain stoichiometric or non-stoichiometric amounts of solvent molecules. “Solvate” encompasses both solution-phase and isolatable solvates. Examples of a solvate include, but are not limited to, hydrates, ethanolates, methanolates, and isopropanolates. Solvation method is well known in the art.

The “stereoisomerism” described in the present invention is divided into conformational isomerism and configurational isomerism. Configurational isomerism can also be divided into cis-trans isomerism and enantiomerism (i.e., optical isomerism). Conformational isomerism refers to a stereoisomerism phenomenon in which each atom or atomic group of an organic molecule with a certain configuration is arranged differently in space due to the rotation or distortion of carbon-carbon single bonds. Common structures having such stereoisomerism phenomenon include alkanes and cycloalkanes, such as the chair conformation and boat conformation appearing in cyclohexane. “Stereoisomer” means that the compound of the present invention contains one or more asymmetric centers, which is thus available as a racemate and a racemic mixture, a single enantiomer, a diastereomeric mixture and a single diastereomers. The compound of the present invention has asymmetric centers, and each asymmetric center can generate two optical isomers. The scope of the present invention includes all possible optical isomers and diastereoisomer mixtures and pure or partially pure compounds.

In particular, the compounds of the present invention can exist in tautomeric forms which have different points of attachment of hydrogens through displacement of one or more double bonds. For example, ketone and enol form thereof are keto-enol tautomers. Each tautomer and a mixture thereof are included in the compounds of the present invention. Enantiomers, diastereoisomers, racemates, mesoisomers, cis-trans isomers, tautomers, geometric isomers, epimers and mixtures thereof of all the compounds are included in the scope of the present invention.

The “isotopic derivative” of the present invention means a molecule of the compound of the present patent that is labeled with an isotope. An isotope commonly used for isotopic labeling includes: hydrogen isotopes, 2H and 3H; carbon isotopes: 11C, 13C and 14C; chlorine isotopes: 35Cl and 37Cl; fluorine isotope: 18F; iodine isotopes: 123I and 125I; nitrogen isotopes: 13N and 15N; oxygen isotopes: 15O, 17O and 18O and sulfur isotope 35S. The isotope-labeled compounds can be used to study the distribution of pharmaceutical molecules in tissues. The substitution with certain heavy isotopes, such as deuterium (2H), can enhance metabolic stability and prolong half-life, thereby achieving the purpose of reducing amounts and providing therapeutic advantages. An isotope-labeled compound is generally synthesized starting from a labeled starting material and using known synthetic techniques in the same way as synthesizing a non-isotope-labeled compound.

The compound of the present invention can be used in combination with an additional SARM1 enzyme activity inhibitor for the treatment or prevention of a neurodegenerative disease or a related neurological disease or condition, or can be used in combination with an additional active drug for the treatment or prevention of a neurodegenerative disease or a related neurological disease or condition, for the treatment or prevention of a neurodegenerative disease or a related neurological disease or condition.

The compound of the present invention or a pharmaceutically acceptable salt thereof can be administered orally or parenterally as an active ingredient, at an effective amount ranging from 0.1 to 2000 mg/kg body weight/day in mammals including humans (weighing about 70 kg), preferably 0.1 to 100 mg/kg body weight/day, in single or divided doses per day, with or without a scheduled time. The administration amount of the active ingredient can be adjusted according to a number of relevant factors such as the condition of the subject to be treated, the type and severity of the disease, the rate of administration and the doctor's opinion. In some cases, an amount less than the above amounts can be appropriate.

The pharmaceutical composition of the present invention can be formulated into a dosage form for oral administration or parenteral administration (including intramuscular, intravenous and subcutaneous routes, and intratumoral injection) according to any one of the conventional methods, where the dosage form is selected from the group consisting of a tablet, a granule, a powder, a capsule, a syrup, an emulsion, a microemulsion, a solution and a suspension.

The pharmaceutical composition of the present invention for oral administration can be prepared by mixing the active ingredient with a carrier selected from the group consisting of cellulose, calcium silicate, magnesium stearate, calcium stearate, corn starch, lactose, sucrose, dextrose, calcium phosphate, stearic acid, a surfactant, a suspending agent, gelatin, talc, an emulsifier and a diluent. Examples of the carrier used in the injectable composition of the present invention include water, glyceride, salt solution, glucose-like solutions, alcohol, glycol, glucose solution, ether (e.g., polyethylene glycol 400), oil, fatty acid, fatty acid ester, a surfactant, a suspending agent and an emulsifier.

Unless otherwise stated, conventional methods of mass spectrometry, nuclear magnetic resonance, HPLC, protein chemistry, biochemistry, recombinant DNA techniques and pharmacology are used. In this application, unless otherwise stated, “or” or “and” refers to “and/or”.

In the specification and claims, a given chemical formula or name shall encompass all stereoisomers and optical isomers, and racemates containing the isomers above. Unless otherwise indicated, all chiral isomers (enantiomers and diastereoisomers) and racemates are within the scope of the present invention. The compounds can comprise many geometric isomers of C═C double bonds, C═N double bonds, ring systems, etc., and all the above stable isomers are encompassed within the present invention. The present invention describes cis- and trans- (or E- and Z-) geometric isomers of the compounds of the present invention, which can be isolated as a mixture of isomers or a separated isomer.

The compounds of the present invention can be isolated in an optically active form or a racemic form. All methods used for preparing the compounds of the present invention and intermediates prepared therein are considered part of the present invention. The enantiomeric or diastereomeric products prepared can be separated by conventional methods, for example by chromatography or fractional crystallization. It should be understood that all tautomeric forms which can exist are included within the present invention. The compounds of the present invention are commercially available as known compounds from the prior art.

Unless otherwise defined, when a substituent is stated as “optionally substituted”, the substituent is selected from the group consisting of, for example, alkyl, cycloalkyl, aryl, heterocyclyl, halogen, hydroxyl, alkoxy, nitro, cyano, oxo, alkanoyl, aryloxy, alkanoyloxy, amino, alkylamino, arylamino, and alkylthio.

The term “alkyl” or “alkylene” as used herein is intended to include both branched and straight chain saturated aliphatic hydrocarbon groups having the indicated number of carbon atoms. In the present invention, alkyl is preferably C₁-C₁₀ alkyl, C₁-C₈ alkyl, more preferably C₁-C₆ alkyl, particularly preferably C₁-C₄ alkyl, especially preferably C₁-C₃ alkyl. For example, “C₁-C₆ alkyl” refers to an alkyl having 1 to 6 carbon atoms. Examples of alkyl include, but are not limited to, methyl (Me), ethyl (Et), propyl (such as n-propyl and isopropyl), butyl (such as n-butyl, isobutyl and t-butyl), and pentyl (such as n-pentyl, isopentyl and neopentyl).

The term “alkoxy” or “alkyloxy” refers to —O-alkyl. For example, “C₁-C₆ alkoxy” (or alkyloxy) is intended to include C₁, C₂, C₃, C₄, C₅, C₆ alkoxy. Preferred alkoxy is C₁-C₁₀ alkoxy, C₁-C₈ alkoxy, more preferably C₁-C₆ alkoxy, particularly preferably C₁-C₄ alkoxy, especially preferably C₁-C₃ alkoxy. Examples of alkoxy include, but are not limited to, methoxy, ethoxy, propoxy (such as n-propoxy and isopropoxy), and tert-butoxy. Similarly, “alkylthio” or “thioalkoxy” represents an alkyl as defined above having the indicated number of carbon atoms and attached through a sulfur bridge, for example, methyl-S— and ethyl-S—. Likewise, preferred alkylthio is C₁-C₁₀ alkylthio, C₁-C₈ alkylthio, more preferably C₁-C₆ alkylthio, particularly preferably C₁-C₄ alkylthio, especially preferably C₁-C₃ alkylthio.

The term “carbonyl” refers to an organic functional group (C═O) formed by the linkage of two atoms, carbon and oxygen, into a double bond.

The term “aryl”, alone or as part of a larger moiety such as “aralkyl”, “aralkoxy” or “aryloxyalkyl”, refers to a monocyclic, bicyclic or tricyclic ring system having a total of 6 to 14 ring members, wherein the system has at least one aromatic ring, and each ring in the system contains 3 to 7 ring members. In certain embodiments of the present invention, “aryl” refers to an aromatic ring system including, but not limited to, phenyl, naphthyl, biphenyl, indanyl, 1-naphthyl, 2-naphthyl, and tetrahydronaphthyl. In the present invention, aryl is preferably C₆-C₁₀ aryl. The term “aralkyl” or “arylalkyl” refers to an alkyl residue attached to an aryl ring. Non-limiting examples thereof include benzyl, phenethyl, and the like.

The term “cycloalkyl” refers to a cyclic alkyl, which can be monocyclic or bicyclic. In the present invention, cycloalkyl is preferably C₃-C₈ cycloalkyl, including but not limited to cyclopropyl, cyclobutyl, cyclopentyl, cyclohexyl and norbornyl.

“Halo” or “halogen” includes fluorine, chlorine, bromine and iodine. “Haloalkyl” is intended to include branched and straight chain saturated aliphatic hydrocarbon groups having the indicated number of carbon atoms and substituted with one or more halogens. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl and heptachloropropyl. Examples of haloalkyl further include fluoroalkyl intended to include branched and straight-chain saturated aliphatic hydrocarbon groups having the indicated number of carbon atoms and substituted with one or more fluorine atoms, particularly preferably trifluoromethyl.

Haloalkoxy refers to a haloalkyl as defined above having the indicated number of carbon atoms and attached through an oxygen bridge. For example, “C₁-C₆ haloalkoxy” is intended to include C₁, C₂, C₃, C₄, C₅, C₆ haloalkoxy. Examples of haloalkoxy include, but are not limited to, trifluoromethoxy, 2,2,2-trifluoroethoxy, and pentafluoroethoxy. Similarly, “haloalkylthio” or “thiohaloalkoxy” refers to a haloalkyl as defined above having the indicated number of carbon atoms and attached through a sulfur bridge; for example trifluoromethyl-S— and pentafluoroethyl —S—.

In the present disclosure, the one or more halogens can each be independently selected from the group consisting of fluorine, chlorine, bromine and iodine.

The term “heteroaryl” refers to a stable 3-, 4-, 5-, 6-, or 7-membered aromatic monocycle, or a 7-, 8-, 9-, 10-membered aromatic bicyclic or polycyclic heterocycle, which is completely unsaturated or partially unsaturated, and contains carbon atoms and 1, 2, 3 or 4 heteroatoms independently selected from the group consisting of N, O and S. Nitrogen and sulfur heteroatoms can be optionally oxidized. Nitrogen atom is substituted or unsubstituted (i.e. N or NR, where R is H or other substituents if defined). A heterocycle can be attached to its side groups at any heteroatom or carbon atom that results in a stable structure. The heterocyclyl described herein can be substituted on carbon or nitrogen atoms if the resulted compound is stable. Nitrogen in the heterocycle can optionally be quaternized. Preferably, in the case that the total number of S and O atoms in the heterocycle exceeds 1, these heteroatoms are not adjacent to each other. Preferably, the total number of S and O atoms in the heterocycle is not greater than one. The term “heterocycle” used is intended to include heteroaryl. Examples of heteroaryl include, but are not limited to, acridinyl, azetidinyl, azocinyl, benzimidazolyl, benzofuryl, benzothiofuryl, benzothienyl, benzoxazolyl, benzoxazolinyl, benzothiazolyl, benzotriazolyl, benzotetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolyl, 2H,6H-1,5,2-dithiazinyl, dihydrofuro[2,3-b]tetrahydrofuryl, furyl, furazanyl, imidazolidinyl, imidazolinyl, imidazolyl, 1H-indazolyl, imidazopyridyl, indolenyl, dihydroindolyl, indolazinyl, indolyl, 3H-indolyl, isatinoyl, isobenzofuryl, isochromanyl, isoindazolyl, isodihydroindolyl, isoindolyl, isoquinolinyl, isothiazolyl, isothiazolopyridyl, isoxazolyl, isoxazolopyridyl, methylenedioxyphenyl, morpholinyl, diazanaphthyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridyl, oxazolidinyl, perimidinyl, oxindolyl, pyrimidinyl, phenanthridinyl, phenanthrolinyl, phenazinyl, phenothiazinyl, phenoxathiyl, phenoxazinyl, phthalazinyl, piperazinyl, piperidinyl, piperidinonyl, 4-piperidinonyl, piperonyl, pteridinyl, purinyl, pyranyl, pyrazinyl, pyrazolidinyl, pyrazolinyl, pyrazolopyridyl, pyrazolyl, pyridazinyl, pyridoxazolyl, pyridoimidazolyl, pyridothiazolyl, pyridyl, pyrimidinyl, pyrrolidinyl, pyrrolinyl, 2-pyrrolidonyl, 2H-pyrrolyl, pyrrolyl, quinazolinyl, quinolinyl, 4H-quinazinyl, quinoxalinyl, quinuclidinyl, tetrazolyl, tetrahydrofuryl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridyl, thienothiazolyl, thienooxazolyl, thienoimidazolyl, thienyl, triazinyl, 1,2,3-triazolyl, 1,2,4-triazolyl, 1,2,5-triazolyl, 1,3,4-triazolyl and xanthenyl, quinolinyl, isoquinolinyl, phthalazinyl, quinazolinyl, indolyl, isoindolyl, dihydroindolyl, 1H-indazolyl, benzoimidazolyl, 1,2,3,4-tetrahydroquinolinyl, 1,2,3,4-tetrahydroisoquinolinyl, 5,6,7,8-tetrahydro-quinolinyl, 2,3-dihydro-benzofuryl, chromanyl, 1,2,3,4-tetrahydro-quinoxalinyl and 1,2,3,4-tetrahydro-quinazolinyl. The term “heteroaryl” can further include biaryl structures formed by the above-defined “aryl” and monocyclic “heteroaryl”, including but not limited to, for example, “-phenylbipyridyl-”, “-phenylbipyrimidyl”, “-pyridylbiphenyl”, “-pyridylbipyrimidyl-” and “-pyrimidylbiphenyl-”; wherein the present invention further includes condensed ring and spiro ring compounds containing, for example, the above heterocycles.

The term “substituted” as used herein means that at least one hydrogen atom is replaced by a non-hydrogen group, provided that normal valences are maintained and that the substitution results in a stable compound. The ring double bond as used herein is a double bond formed between two adjacent ring atoms (e.g., C═C, C═N or N═N).

When any variable occurs more than once in any composition or formula of a compound, its definition on each occurrence is independent of its definition at every other occurrence. Therefore, for example, if a group is shown to be substituted with 0-3 R, the group can be optionally substituted with up to three R groups, and R at each occurrence is independently selected from the definition of R. In addition, a combination of substituents and/or variables is permissible only if such combination results in a stable compound.

The term “effective amount” as used herein refers to an amount of a drug or an agent (i.e., the compound of the invention) that will elicit, for example, a biological or medical response in a tissue, system, animal or human being sought by a researcher or clinician. Furthermore, the term “therapeutically effective amount” refers to an amount that results in improved treatment, cure, prevention or alleviation of a disease, condition or side effects, or a reduction in the progression rate of a disease or condition compared to corresponding subjects without receiving the above amounts. An effective amount can be given in one or more administrations, applications or doses, and is not intended to be limited by a particular formulation or route of administration. The term also includes an effective amount within its scope that enhances normal physiological function.

The term “treating” as used herein includes any effects of ameliorating a condition, disease, disorder, etc., such as alleviating, reducing, regulating, ameliorating or eliminating a condition, disease or disorder, or ameliorating the symptoms thereof.

The term “pharmaceutically acceptable” used herein refers to a compound, a substance, a composition and/or a dosage form which, within the scope of rational medical judgment, is suitable for use in contact with human and animal tissues, without undue toxicity, irritation, allergic reaction and/or other problems or complications, and commensurates with a reasonable benefit/risk ratio.

As used herein, the phrase “pharmaceutically acceptable carrier” refers to a pharmaceutically acceptable substance, composition or vehicle, such as a liquid or solid filler, a diluent, an excipient, a manufacturing aid (e.g. a lubricant, talc, magnesium stearate, calcium stearate, zinc stearate, or stearic acid) or a solvent-encapsulated substance, which relates to carrying or transporting a subject compound from one organ or body part to another. Each carrier must be “acceptable” in the sense of being compatible with the other ingredients of the formulation and not harmful to the patient.

The term “pharmaceutical composition” refers to a composition comprising the compound of the present invention and at least one additional pharmaceutically acceptable carrier. “Pharmaceutically acceptable carrier” refers to a medium generally used in the art for the delivery of a biologically active agent to an animal, particularly a mammal, including (i.e.) an adjuvant, an excipient or a vehicle, such as a diluent, a preservative, a filler, a flow regulator, a disintegrant, a wetting agent, an emulsifier, a suspending agent, a sweetener, a flavoring agent, a fragrance, an antibacterial agent, an antifungal agent, a lubricant and a dispersant, depending on the mode of administration and property of dosage form.

As used herein, the administration of a certain compound or pharmaceutical composition can ameliorate a certain disease, symptom or condition, especially alleviating the severity thereof, delaying the onset, slowing down the progression of the disease, or reducing the duration of the disease. The effects can be attributed to or related to the administration, whether it is regular or temporary administration, continuous or intermittent administration.

Route of Administration

Suitable routes of administration include, but are not limited to, oral administration, intravenous injection, rectal administration, aerosol spray, parenteral administration, ocular administration, pulmonary administration, transdermal administration, vaginal administration, ear canal administration, nasal administration and topical administration. In addition, only for example, parenteral administration includes intramuscular injection, subcutaneous injection, intravenous injection, intramedullary injection, intraventricular injection, intraperitoneal injection, intralymphatic injection, and intranasal injection.

In one aspect, the compounds described herein are administered locally rather than systemically. In certain embodiments, the long-acting formulation is administered by implantation (e.g., subcutaneously or intramuscularly) or by intramuscular injection. Additionally, in another specified embodiment, the drug is administered via a targeting drug delivery system, for example, a liposome encapsulated with organ-specific antibodies. In such specified embodiment, the liposome is selectively directed to and absorbed by the specific organ.

In the pharmaceutical composition of the present invention, the pharmaceutically acceptable carrier can be formulated according to various factors within the knowledge of those skilled in the art. These factors include, but are not limited to, the type and property of the active agent to be formulated; the subject to be administered with the composition comprising the active agent; the intended route of administration of the composition; and the targeting therapeutic indication. The pharmaceutical carrier includes aqueous and non-aqueous liquid media and various solid and semisolid dosage forms.

The above carriers can include various ingredients and additives other than the active agent, and the above additional ingredients, such as a stabilizing active agent and a binding agent, are included in the formulation for various reasons known to those skilled in the art. Description of suitable pharmaceutical carriers and the factors involved in carrier selection can be found in several readily available sources, such as Allen L. V. Jr. et al. Remington: The Science and Practice of Pharmacy (2 Volumes), 22^{nd} Edition (2012), Pharmaceutical Press.

The compound is generally administered in a mixture form with a suitable pharmaceutical diluent, excipient or carrier (collectively referred to herein as a drug carrier), which is appropriately selected according to the intended form of administration (e.g., oral tablets, capsules, elixirs and syrups) and consistent with conventional pharmaceutical practice.

Although the compound of the present invention can be administered alone, it is preferably administered in the form of a pharmaceutical formulation (composition).

Kit/Product Packaging

Kit/product packaging is also described herein for the treatment of the above indications. The kit can consist of a transporter, a drug pack, or a container box. The container box can be divided into compartments to accommodate one or more types of containers, such as a vial, a test tube, and the like. Each container contains a single component in the method. A suitable container includes a bottle, a vial, a syringe, a test tube and the like. The container is made of acceptable materials such as glass or plastic.

For example, a container can contain one or more compounds described herein. The compound exists either as a pharmaceutical composition or in a mixture with other ingredients described herein. The container can have a sterile outlet (e.g., the container can be an intravenous infusion pack or bottle, the stopper of the container can be pierced by a hypodermic needle). Such kit can contain a compound, an instruction for use, a label or an instruction for operation described herein.

A typical kit can include one or more containers. Each container contains one or more materials (such as a reagent, or a concentrated stock solution thereof, and/or an equipment) to meet the needs of commercial promotion and requirements of the use of compounds by users. These materials include, but are not limited to, a buffer, a diluent, a filter, a needle, a syringe, a transporter, a bag, a container, a bottle and/or a test tube, accompanied by a list of contents and/or an instruction for use, and the inner packaging is also accompanied by an instruction. Instructions for the entire kit should be included.

The above features mentioned in the present invention, or the features mentioned in the embodiments can be combined arbitrarily. All the features disclosed in the specification of this application can be used in combination with any composition, and each feature disclosed in the specification can be replaced by any alternative feature that can provide the same, equivalent or similar purpose. Therefore, unless otherwise specified, the disclosed features are only general examples of equivalent or similar features.

The present invention is further illustrated below in conjunction with specific examples. It should be understood that these examples are only used to illustrate the present invention and are not intended to limit the scope of the present invention. The experimental methods without specific conditions indicated in the following examples are usually conducted following the conventional conditions or the conditions suggested by the manufacturer. Unless otherwise indicated, all percentages, ratios, proportions, or parts are calculated by weight.

The unit of weight volume percentage in the present invention is well known to those skilled in the art, for example, it refers to the weight of solute in 100 ml of solution. Unless otherwise defined, all professional and scientific terms used herein have the same meanings as commonly understood by those skilled in the art. In addition, any methods and materials similar or equivalent to the content described herein can be applied to the method of the present invention. The preferred embodiments and materials described herein are for demonstration purposes only.

### EXAMPLES

The present teachings include descriptions provided in the examples, which are not intended to limit the scope of any claims. The following non-limiting examples are provided to further illustrate the present invention. According to the present disclosure, those skilled in the art will appreciate that, without departing from the spirit and scope of the teachings, multiple changes can be made to the specific embodiments disclosed, and the same or a similar result can still be obtained.

The compounds tested for SARM 1 enzyme activity were obtained form 1) a “Marketed Drugs” compound library for screening (purchased from TargetMol, approximately 2000 compounds); 2) compounds purchased individually, or 3) compounds (I), (II), and (III) synthesized using the following general method.

### Example 1: Synthesis of Compound Represented by Formula I

The compound represented by formula I was synthesized according to the following synthetic method: a compound represented by formula I-1 and a compound represented by formula I-2 were subjected to a cyclization reaction in the presence of 1) sulfuryl chloride and 2) oxygen.

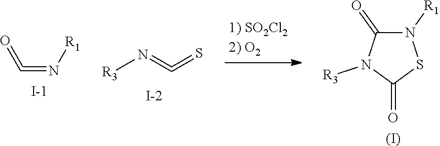

### Example 2: Synthesis of Compound Represented by Formula II-a

The compound represented by formula II-a was synthesized according to the following synthetic method: a compound represented by formula II-1 and a compound represented by formula II-2 were subjected to a reaction in the presence of a base to produce a compound represented by formula II-3, and the compound represented by formula II-3 was subjected to a cyclization reaction in the presence of bromine.

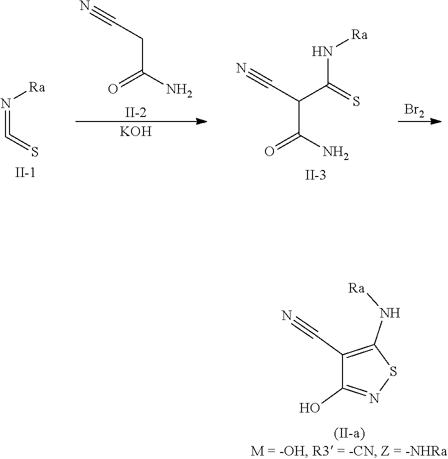

### Example 3: Synthesis of Compound Represented by Formula III

The compound represented by formula III was synthesized according to the following synthesis method: a compound represented by formula III-1 and a compound represented by formula III-2 were subjected to a cyclization reaction.

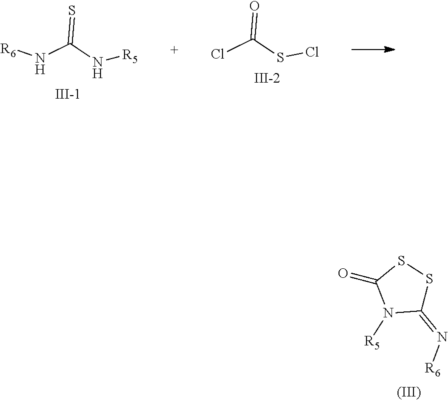

### BIOLOGICAL ACTIVITY EXAMPLES

### Example 4: Preparation of SARM1 and NAD Enzyme Activity Test

Preparation of Test Compounds:

The stock solutions of test compounds had concentrations of 200 μM or 10 mM (in DMSO), which were further diluted to desired compound concentrations for in vitro SARM1 enzyme assays and screening of inhibitors.

Expression and Purification of SARM1 Protein

(1) Construction of Plasmid

In this example, dN-SARM1 gene sequence was amplified by PCR. The mitochondrial localization signal peptide at N-terminal of SARM1 was removed. The PCR amplification product was constructed into the pLenti-CMV-puro-dest plasmid (addgene catalog #17452), as follows:

BC2T-TEV polypeptide gene fragment, dN-SARM1-F and dN-SARM1-R were synthesized by Shanghai Sangon Company. BC2T-TEV polypeptide gene fragment had a sequence set forth in Seq ID No.1, dN-SARM1-F had a sequence set forth in Seq ID No.2, and dN-SARM1-R had a sequence set forth in Seq ID No.3.

Seq ID No. 1:
5′-CTCATGccagacagaaaagcggctgttagtcactggcagcaaGATAT
CGGCGGAGGCGGATCTGGCGGAGGCGGATCTGGCGGAGGCGGATCTgaga
atttgtattttcagggtGGCGGAGGCGGAGGTACCCTG-3′
Seq ID No. 2:
5′-GGTACCCTGGCGGTGCCTGGGCCAG-3′
Seq ID No. 3:
5′-GCGGCCGCCTAGGTTGGACCCATGGGTGCAGCACCC-3′

The synthetic BC2T-TEV polypeptide gene fragment was ligated to pENTR vector pENTR1A-GFP-N2 (addgene: catalog #19364) using HindIII/KpnI restriction sites. The dN-SARM1 gene fragment was amplified with primers dN-SARM1-F and dN-SARM1-R, and the dN-SARM1 gene fragment obtained by amplification was constructed into pENTR vector with BC2T-TEV through KpnI and NotI restriction sites. All endonucleases used in this example were purchased from thermo.

The dN-SARM1 gene fragment obtained by PCR amplification had a sequence set forth in Seq ID No.4.

Seq ID No. 4:
GGTACCCTGGCGGTGCCTGGGCCAGATGGGGGCGGTGGCACGGGCCCATG
GTGGGCTGCGGGTGGCCGCGGGCCCCGCGAAGTGTCGCCGGGGGCAGGCA
CCGAGGTGCAGGACGCCCTGGAGCGCGCGCTGCCGGAGCTGCAGCAGGCC
TTGTCCGCGCTGAAGCAGGCGGGCGGCGCGCGGGCCGTGGGCGCCGGCCT
GGCCGAGGTCTTCCAACTGGTGGAGGAGGCCTGGCTGCTGCCGGCCGTGG
GCCGCGAGGTAGCCCAGGGTCTGTGCGACGCCATCCGCCTCGATGGCGGC
CTCGACCTGCTGTTGCGGCTGCTGCAGGCGCCGGAGTTGGAGACGCGTGT
GCAGGCCGCGCGCCTGCTGGAGCAGATCCTGGTGGCTGAGAACCGAGACC
GCGTGGCGCGCATTGGGCTGGGCGTGATCCTGAACCTGGCGAAGGAACGC
GAACCCGTAGAGCTGGCGCGGAGCGTGGCAGGCATCTTGGAGCACATGTT
CAAGCATTCGGAGGAGACATGCCAGAGGCTGGTGGCGGCCGGCGGCCTGG
ACGCGGTGCTGTATTGGTGCCGCCGCACGGACCCCGCGCTGCTGCGCCAC
TGCGCGCTGGCGCTGGGCAACTGCGCGCTGCACGGGGGCCAGGCGGTGCA
GCGACGCATGGTAGAGAAGCGCGCAGCCGAGTGGCTCTTCCCGCTCGCCT
TCTCCAAGGAGGACGAGCTGCTTCGGCTGCACGCCTGCCTCGCAGTAGCG
GTGTTGGCGACTAACAAGGAGGTGGAGCGCGAGGTGGAGCGCTCGGGCAC
GCTGGCGCTCGTGGAGCCGCTTGTGGCCTCGCTGGACCCTGGCCGCTTCG
CCCGCTGTCTGGTGGACGCCAGCGACACAAGCCAGGGCCGCGGGCCCGAC
GACCTGCAGCGCCTCGTGCCGTTGCTCGACTCTAACCGCTTGGAGGCGCA
GTGCATCGGGGCTTTCTACCTCTGCGCCGAGGCTGCCATCAAGAGCCTGC
AAGGCAAGACCAAGGTGTTCAGCGACATCGGCGCCATCCAGAGCCTGAAA
CGCCTGGTTTCCTACTCTACCAATGGCACTAAGTCGGCGCTGGCCAAGCG
CGCGCTGCGCCTGCTGGGCGAGGAGGTGCCACGGCCCATCCTGCCCTCCG
TGCCCAGCTGGAAGGAGGCCGAGGTTCAGACGTGGCTGCAGCAGATCGGT
TTCTCCAAGTACTGCGAGAGCTTCCGGGAGCAGCAGGTGGATGGCGACCT
GCTTCTGCGGCTCACGGAGGAGGAACTCCAGACCGACCTGGGCATGAAAT
CGGGCATCACCCGCAAGAGGTTCTTTAGGGAGCTCACGGAGCTCAAGACC
TTCGCCAACTATTCTACGTGCGACCGCAGCAACCTGGCGGACTGGCTGGG
CAGCCTGGACCCGCGCTTCCGCCAGTACACCTACGGCCTGGTCAGCTGCG
GCCTGGACCGCTCCCTGCTGCACCGCGTGTCTGAGCAGCAGCTGCTGGAA
GACTGCGGCATCCACCTGGGCGTGCACCGCGCCCGCATCCTCACGGCGGC
CAGAGAAATGCTACACTCCCCGCTGCCCTGTACTGGTGGCAAACCCAGTG
GGGACACTCCAGATGTCTTCATCAGCTACCGCCGGAACTCAGGTTCCCAG
CTGGCCAGTCTCCTGAAGGTGCACCTGCAGCTGCATGGCTTCAGTGTCTT
CATTGATGTGGAGAAGCTGGAAGCAGGCAAGTTCGAGGACAAACTCATCC
AGAGTGTCATGGGTGCCCGCAACTTTGTGTTGGTGCTATCACCTGGAGCA
CTGGACAAGTGCATGCAAGACCATGACTGCAAGGATTGGGTGCATAAGGA
GATTGTGACTGCTTTAAGCTGCGGCAAGAACATTGTGCCCATCATTGATG
GCTTCGAGTGGCCTGAGCCCCAGGTCCTGCCTGAGGACATGCAGGCTGTG
CTTACTTTCAACGGTATCAAGTGGTCCCACGAATACCAGGAGGCCACCAT
TGAGAAGATCATCCGCTTCCTGCAGGGCCGCTCCTCCCGGGACTCATCTG
CAGGCTCTGACACCAGTTTGGAGGGTGCTGCACCCATGGGTCCAACCTAG

The PCR amplification reaction system was as follows: 5×PrimeSTAR Buffer (Mg²⁺ plus) 10 μL and dNTP Mixture (2.5 mM each) 4 μL were added with dN-SARM1-F at a final concentration of 0.2 μmol/L, dN-SARM1-R at a final concentration of 0.2 μmol/L, 100 ng of DNA template, and 0.5 μL of PrimeSTAR HS DNA Polymerase (2.5 U/μL), and the mixture was finally added with sterilized ddH₂O to 50 μL. The full-length SARM1 was fully synthesized into the pUC57 plasmid by WZ Bioscience Inc., which was subjected to PCR using pUC57-SARM1 as a DNA template.

The PCR amplification product was subjected to agarose gel electrophoresis, and then recovered and purified with Omega Gel Recovery Kit D2500-02. The specific steps of gel cutting and recovery were referred to the kit instruction. The recovered and purified PCR amplification product was constructed into the pENTR vector with BC2T-TEV

A recombinant plasmid was constructed by the steps as follows:

Enzymatic digestion reaction system: 800 ng of PCR amplification product or plasmid, 1p L of endonuclease (Fastdigest), and 1 μL of buffer solution were supplemented with sterilized water to a volume of 10 μL. The enzyme digestion reaction was performed at a constant temperature of 37° C. for 30 min.

Plasmid ligation: After the enzyme digestion reaction was completed, 300 ng of the enzyme-digested PCR amplification product and 50 ng of the enzyme-digested plasmid were mixed with 1 μL of T4 DNA ligase and 1 μL of T4 DNA ligase buffer solution evenly, and the mixture was added with sterilized water to volume 20 μL. The ligation was carried out at a constant temperature of 16° C. overnight.

The ligated product was subjected to agarose gel electrophoresis, and then recovered and purified with Omega Gel Recovery Kit D2500-02. The recovered and purified product was the recombinant plasmid of this example, labeled as pENTR1A-BC2T-dN-SARM1.

After the pENTR1A-BC2T-dN-SARM1 plasmid was constructed, dN-SARM1 was recombined into pLenti-CMV-puro-dest by LR reaction.

Recombination reaction system: 150 ng of pENTR1A-BC2T-dN-SARM1, 50 ng of pLenti-CMV-puro-dest and 1 μL of 5× LR Clonase™ reaction buffer were supplemented with sterile water to a total volume of 5 μL.

(2) Transfection

In this example, the constructed pLenti-CMV-puro-dest and viral packaging plasmids psPAX2 and pMD2.G (addgene psPAX2: #12260, pMD2.G: #12259) were co-transfected into HEK293T cells (ATCC) by liposome lipofectamine 2000 (Life Technologies) to prepare viruses with dN-SARM1 reading frame. The specific steps are as follows:

1×10⁶ cells were plated in a 3.5 cm dish and transfected the next day.

Plasmid mixture: The transfection was conducted with 1.7 μg of pLenti-dN-SARM1, 1.7 μg of psPAX2, 0.6 μg of pMD2.G and 8 μL of lipofectamine 2000 transfection reagent according to the instruction. After 8 hours, the medium was changed, and the viruses for 48 hours were collected.

(3) Cell Screening

HEK293T cells obtained in step “(2) Transfection” were infected with dN-SARM1 virus, and added with puromycin for screening to obtain cells stably expressing dN-SARM1 protein. The specific steps are as follows:

Virus: 80 μL/3.5 cm of cells were infected with 2×10⁵ viruses for 48 h, and then added with 2 μg/mL of puromycin for 48 h of screening. The cells that were not infected with the virus were completely dead. Most of the virus-infected cells survived, and were added with 2 μg/mL puromycin for 48 h of secondary screening.

(4) Protein Extraction

The cells stably expressing dN-SARM1 protein obtained in step “(3) Cell screening” were cultured, collected, and subjected to digitonin cleavage to obtain the dN-SARM1 protein expressed in the cytoplasm for in vitro activity assay. The specific steps are as follows:

The cells were cultured in 10 cm dish with DMEM, digested with trypsin-EDTA, then centrifuged at 1000 rpm for 5 min, washed once with PBS, and then resuspended with PBS containing 100 μM digitonin at 0.6 mL PBS/10 cm cells for 5 min of lysis. The cells were added with trypan blue and observed under a microscope. More than 90% of the cells were lysed. Then the cells were centrifuged at 5000 rpm for 10 min to collect the supernatant of dN-SARM1 protein.

### Example 5: In Vitro Biochemical Test of Inhibiting SARM1 Enzyme Activity (% Inhibition Rate)

The compounds were detected by PC6 fluorescence method [Chinese patent 202010528147.3] with the dN-SARM1 protein obtained in “Expression and purification of SARM1 protein” and “(4) Protein extraction” in Example 4 above.

Reaction Conditions:

First, 0.05 μg/ml dN-SARM1 and 50 μM compound were incubated in 50 mM Tris-HCl (pH 7.5) solution for 10 min. Then 50 μM NAD, 50 μM PC6 as substrate and 50 μM NMN as activator were added to the dN-SARM1 protein incubated with the drug for 30 min of reaction at room temperature. The concentration of each component was the final concentration in the reaction system.

During the reaction process, PC6 fluorescence spectrum kinetics was detected by a microplate reader at an excitation wavelength and an emission wavelength of 390 nm and 520 nm, respectively. Finally, the activity of the protein was denoted by the reaction rate. The higher the reaction rate, the stronger the protein activity, and the lower the inhibition efficiency of the compound.

The inhibition rates of SARM1 enzyme activity by some compounds at 50 μM are provided in Table 1 below:

TABLE 1

| | Inhibition rate of |
|---|---|
| Compound number | enzyme activity |
| 7 | 98.6% |
| 32 | 93.7% |
| 35 | 102% |
| 36 | 98.5% |
| 37 | 101% |
| 40 | 75.6 |
| 41 | 67.3% |
| 42 | 67.4% |
| 43 | 85.3% |

### Example 6: In Vitro Biochemical Test of Inhibiting SARM1 Enzyme Activity (IC₅₀)

First, 200 μM of the compound was added to a 50 mM Tris-HCl (pH 7.5) solution containing 0.05 μg/ml dN-SARM1. Then half of the mixture was added to an equal volume of 50 mM Tris-HCl (pH 7.5) solution containing 0.05 μg/ml dN-SARM1 (pH 7.5) for mixing. The drug was diluted 6 times by analogy to final concentrations of 200, 100, 50, 25, 12.5, 6.25, 3.125 μM, or 200, 50, 12.5, 3.125, 0.78, 0.195, 0.049 μM. The solution without addition of inhibitor was control group. The above solutions were incubated at room temperature for 10 min.

Then 50 μM NAD, 50 μM PC6 as substrate and 50 μM NMN as activator were added to the dN-SARM1 protein incubated with inhibitor for 30 min of reaction at room temperature. The concentration of each component was the final concentration in the reaction system.

During the reaction process, PC6 fluorescence spectrum kinetics was detected by a microplate reader at an excitation wavelength and an emission wavelength of 390 nm and 520 nm, respectively. Finally, the activity of the protein was denoted by the reaction rate, and the half-inhibition concentration was calculated. The higher the reaction rate, the stronger the protein activity, and the lower the inhibition efficiency of the compound.

The amount curves of the compounds inhibiting SARM1 enzyme activity were as described above.

IC₅₀ ranges for these compounds in the assay are provided in Table 2 below:

IC₅₀ ranges for inhibiting SARM1 enzyme activity: A<1.0 μM; B: 1-10 μM; C:>10 μM

TABLE 2

| Compound number | IC₅₀ (PC6) |
|---|---|
| 1 | A |
| 2 | A |
| 3 | A |
| 4 | A |
| 5 | A |
| 6 | A |
| 7 | A |
| 8 | B |
| 11 | B |
| 12 | C |
| 13 | B |
| 14 | C |
| 15 | A |
| 17 | C |
| 18 | C |
| 24 | A |
| 32 | C |
| 35 | C |
| 36 | C |
| 37 | A |
| 39 | B |
| 43 | C |

### Example 7: Inhibitory Activity of Drugs Detected in Cell Lines with Inducible Overexpression of SARM1

(1) Preparation of iSARM1 Cell Line

In this example, the gene sequence of SARM1 was amplified by PCR and constructed into the pInducer20-neo plasmid. The pInducer20-SARM1 virus was packaged in liposomes, which then infected HEK293 to obtain a cell line with inducible overexpression of SARM1, labeled as iSARM1 (HEK293). The specific steps are as follows:

In this example, the SARM1 gene sequence was amplified by PCR with the primers of the sequences set forth in Seq ID No.5 and Seq ID No.6. The recovery of PCR amplification products, enzyme digestion, construction of recombinant plasmids, transfection and cell screening were all the same as that of the dN-SARM1 in “Expression and purification of SARM1 protein”, except that in “(3) Cell screening”, “2 μg/mL puromycin” was replaced by 2 mg/mL neomycin, and will not be elaborated here.

Seq ID No. 5:
5′-TCTAGAGCCACCATGGTCCTGACGCTGCTTC-3′
Seq ID No. 6:
5′-GAATTCTTAGGTTGGACCCATGGGTG-3′

(2) Detection of the Inhibition of the Activity of SARM1 Protein by Inhibitors in Cell Lines

Firstly, a 96-well culture dish was treated with 0.05 mg/ml polylysine for 5 min, and washed once with PBS. 3×10⁴ iSARM1 (HEK293) cells were plated into the 96-well plate and cultured in a 37° C. and 5% incubator overnight. The next day, the cells were added with an inhibitor with a final concentration of 50 μM, cultured in the incubator for 1.5 h, then added with the activator CZ-48 with a final concentration of 100 μM, and co-cultured for 16 h. In addition, a control group without addition of CZ-48 or drug was set up. Finally, the intracellular cADPR level was detected to denote the activity of SARM1, and the inhibition rate of SARM1 by 50 μM inhibitor in cells was calculated.

The cADPR assay was conducted as follows. First, the cells were washed once with PBS, and added with 150 μl of pre-cooled 0.6M perchloric acid (PCA) for rapid lysis of the cells and precipitation of proteins. The PCA supernatant was transferred to a 1.5 ml centrifuge tube, and the protein in the medium was redissolved with 100 μl of 1M NaOH. 0.5 ml of an organic reagent mixture (trioctylamine:chloroform=1:3) was added to the supernatant to extract PCA from water. After sufficient shaking, the mixture was centrifuged at 12,000 rpm for 10 min, and the solution was divided into three layers: the upper aqueous phase, containing the target small molecule; the lower organic phase, in which PCA is dissolved; and a thin layer of protein between the upper and lower layers. The upper layer was transferred to a new centrifuge tube. The solution was added with 1M Tris-Mg (1M Tris (pH 8.0): 1M MgCl2=9:1) at a ratio of 1:100, and added with NADase at a ratio of 1:250. The mixed solution was treated at 37° C. overnight to remove the NAD⁺ from the mixed solution. After the treatment was completed, the mixed solution was filtered with a Millipore 10 K 96-well filter plate to remove NADase.

The content of cADPR in the solution was determined by Cycling analysis, which was operated as follows. 20 μl of the sample to be tested or the cADPR standard were added into a 96-well opaque white plate. Preparation of reaction solution: 9.6 ml of PBS (pH 7.4), 200 μl of ethanol, 150 μl of 1 mg/ml AD, 10 μl of 10 mM FMN, 5 μl of 18 mg/ml Diaphorase, 10 μl of 10 mM Resazurin, and 100 μl of 1M Nam were mixed. Half of the reaction solution was added into 0.2 μg/ml cyclase, and the reaction solution without addition of cyclase was used as a control experiment. Each sample was divided into two groups, 3 replicates in each group. Each well of the 96-well plate was added with the reaction solution containing or not containing cyclase for reaction, and the reaction kinetic curve within 30 min (Ex:Em=544/599) was recorded. The average slope of the reaction was calculated, and the accurate cADPR content was calculated by converting the cADPR standard.

Calculation method of inhibition rate: (1-cADPR content of inhibitor group/cADPR content of control group)*100%

For the above method, the inhibition rates of the drugs in the cell line with inducible overexpression of SARM1 are shown in Table 3 below: Cell activity inhibition rate range: A>50%; B: 25-50%; C:<25%

TABLE 3

| Compound number | Inhibition rate |
|---|---|
| 2 | B |
| 11 | A |
| 24 | A |

1. A method for the treatment or prevention of a neurodegenerative disease or a neurological disease or condition, comprising administering an SARM1 enzyme activity inhibitor to a subject in need thereof;
wherein the SARM1 enzyme activity inhibitor is a compound represented by formula I: 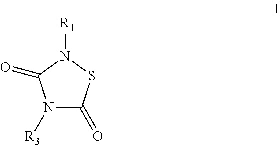
wherein, R₁ and R₃ are independently selected from the group consisting of: hydrogen, C₁-C₁₀ alkyl, C₃-C₈ cycloalkyl, C₆-C₁₀ aryl, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl; wherein the C₁-C₁₀ alkyl, C₃-C₈ cycloalkyl, C₆-C₁₀ aryl, C₆-C₁₀ arylamino, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl are optionally substituted by 1, 2 or 3 substituents selected from the group consisting of halogen selected from the group consisting of fluorine, chlorine, bromine and iodine, nitro, cyano, C₁-C₃ alkyl, C₁-C₃ alkoxy, halogenated C₁-C₃ alkyl, halogenated C₁-C₃ alkylthio, and C₃-C₈ cycloalkyl C₁-C₃ alkyl.

2. A method for the treatment or prevention of a disease or condition related to axonal degeneration, comprising administering an SARM1 enzyme activity inhibitor to a subject in need thereof;
wherein the SARM1 enzyme activity inhibitor is a compound represented by formula I: 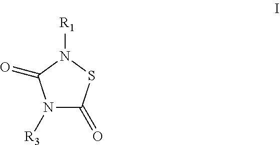
wherein, R₁ and R₃ are independently selected from the group consisting of: hydrogen, C₁-C₁₀ alkyl, C₃-C₈ cycloalkyl, C₆-C₁₀ aryl, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl; wherein the C₁-C₁₀ alkyl, C₃-C₈ cycloalkyl, C₆-C₁₀ aryl, C₆-C₁₀ arylamino, C₆-C₁₀ aryl C₁-C₃ alkyl, C₆-C₁₀ heteroaryl, C₆-C₁₀ heteroaryl C₁-C₃ alkyl, C₁-C₃ alkoxy, C₁-C₃ alkylamino, C₁-C₃ alkylthio, C₁-C₃ alkylsulfonyl, C₁-C₃ alkylacyl, C₁-C₃ alkylaminoacyl and C₁-C₃ alkylaminosulfonyl are optionally substituted by 1, 2 or 3 substituents selected from the group consisting of halogen selected from the group consisting of fluorine, chlorine, bromine and iodine, nitro, cyano, C₁-C₃ alkyl, C₁-C₃ alkoxy, halogenated C₁-C₃ alkyl, halogenated C₁-C₃ alkylthio, and C₃-C₈ cycloalkyl C₁-C₃ alkyl.

3. The method according to claim 1, wherein the neurodegenerative disease or the neurological disease or condition is selected from the group consisting of Alzheimer's disease, Parkinson's disease, multiple sclerosis, amyotrophic lateral sclerosis and peripheral neuropathy.

4. (canceled)

5. (canceled)

6. (canceled)

7. (canceled)

8. (canceled)

9. The method according to claim 1, wherein R₁ and R₃ are each independently selected from the group consisting of: C₁-C₃ alkyl; phenyl, benzyl and naphthyl, which are optionally substituted by substituents selected from the group consisting of methyl, isopropyl, trifluoromethyl, fluoro, chloro and nitro; cyclopropylmethyl; cyano and hydroxyl.

10. The method according to claim 1, wherein R₁ and R₃ are each independently selected from the group consisting of: methyl, benzyl, phenyl, naphthyl, p-methylphenyl, p-fluorophenyl, isopropylphenyl, trifluoromethylthiophenyl, nitro-, methyl- or chloro-substituted phenyl, cyclopropylmethyl, trifluoromethyl-substituted phenyl.

11. The method according to claim 1, wherein the compound is selected from the group consisting of the following compounds, pharmaceutically acceptable salts and prodrugs thereof:

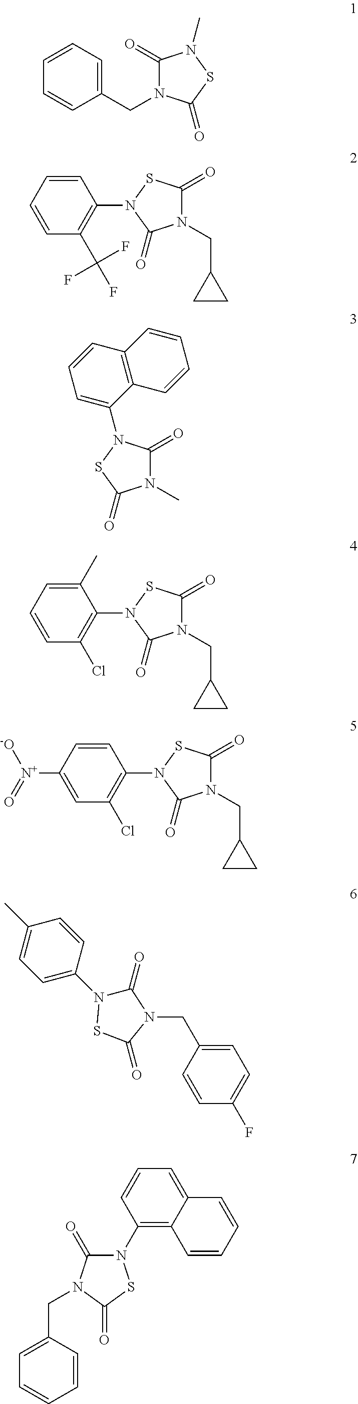

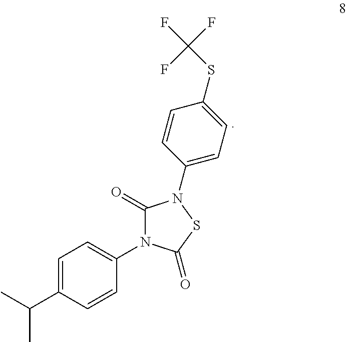 [SMILES: CC(C)c1ccc(-n2c(=O)sn(-c3ccc(SC(F)(F)F)cc3)c2=O)cc1]

12. (canceled)

13. (canceled)

14. (canceled)

15. (canceled)

16. (canceled)

17. (canceled)

18. The method according to claim 2, wherein the disease or condition related to axonal degeneration is selected from the group consisting of Alzheimer's disease, Parkinson's disease, multiple sclerosis, amyotrophic lateral sclerosis and peripheral neuropathy.

19. The method according to claim 2, wherein R₁ and R₃ are each independently selected from the group consisting of: C₁-C₃ alkyl; phenyl, benzyl and naphthyl, which are optionally substituted by substituents selected from the group consisting of methyl, isopropyl, trifluoromethyl, fluoro, chloro and nitro; cyclopropylmethyl; cyano and hydroxyl.

20. The method according to claim 2, wherein R₁ and R₃ are each independently selected from the group consisting of: methyl, benzyl, phenyl, naphthyl, p-methylphenyl, p-fluorophenyl, isopropylphenyl, trifluoromethylthiophenyl, nitro, methyl- or chloro-substituted phenyl, cyclopropylmethyl, trifluoromethyl-substituted phenyl.

21. The method according to claim 2, wherein the compound is selected from the group consisting of the following compounds, pharmaceutically acceptable salts and prodrugs thereof: 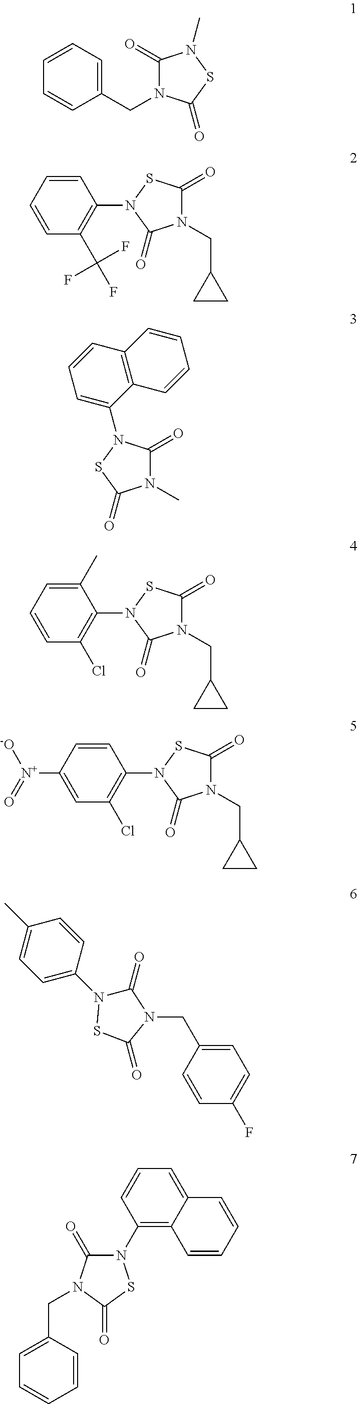 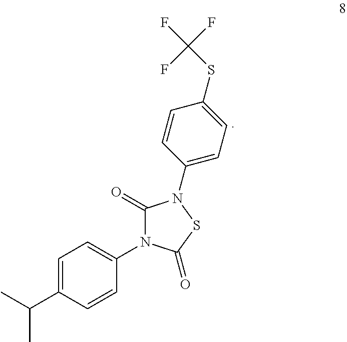 [SMILES: CC(C)c1ccc(-n2c(=O)sn(-c3ccc(SC(F)(F)F)cc3)c2=O)cc1]


SEQUENCE LISTING (6 sequences)

SEQ ID NO: 1 - DNA, 135 bp, Artificial Sequence
  BC2T-TEV polypeptide gene fragment
ctcatgccagacagaaaagcggctgttagtcactggcagcaagatatcggcggaggcggatctggcggaggcggatctggcggaggcggatctgagaatttgtattttcagggtggcggaggcggaggtaccctg

SEQ ID NO: 2 - DNA, 25 bp, Artificial Sequence
  Primer dN-SARM1-F
ggtaccctggcggtgcctgggccag

SEQ ID NO: 3 - DNA, 36 bp, Artificial Sequence
  Primer dN-SARM1-R
gcggccgcctaggttggacccatgggtgcagcaccc

SEQ ID NO: 4 - DNA, 2100 bp, Artificial Sequence
  dN-SARM1 gene fragment
ggtaccctggcggtgcctgggccagatgggggcggtggcacgggcccatggtgggctgcgggtggccgcgggccccgcgaagtgtcgccgggggcaggcaccgaggtgcaggacgccctggagcgcgcgctgccggagctgcagcaggccttgtccgcgctgaagcaggcgggcggcgcgcgggccgtgggcgccggcctggccgaggtcttccaactggtggaggaggcctggctgctgccggccgtgggccgcgaggtagcccagggtctgtgcgacgccatccgcctcgatggcggcctcgacctgctgttgcggctgctgcaggcgccggagttggagacgcgtgtgcaggccgcgcgcctgctggagcagatcctggtggctgagaaccgagaccgcgtggcgcgcattgggctgggcgtgatcctgaacctggcgaaggaacgcgaacccgtagagctggcgcggagcgtggcaggcatcttggagcacatgttcaagcattcggaggagacatgccagaggctggtggcggccggcggcctggacgcggtgctgtattggtgccgccgcacggaccccgcgctgctgcgccactgcgcgctggcgctgggcaactgcgcgctgcacgggggccaggcggtgcagcgacgcatggtagagaagcgcgcagccgagtggctcttcccgctcgccttctccaaggaggacgagctgcttcggctgcacgcctgcctcgcagtagcggtgttggcgactaacaaggaggtggagcgcgaggtggagcgctcgggcacgctggcgctcgtggagccgcttgtggcctcgctggaccctggccgcttcgcccgctgtctggtggacgccagcgacacaagccagggccgcgggcccgacgacctgcagcgcctcgtgccgttgctcgactctaaccgcttggaggcgcagtgcatcggggctttctacctctgcgccgaggctgccatcaagagcctgcaaggcaagaccaaggtgttcagcgacatcggcgccatccagagcctgaaacgcctggtttcctactctaccaatggcactaagtcggcgctggccaagcgcgcgctgcgcctgctgggcgaggaggtgccacggcccatcctgccctccgtgcccagctggaaggaggccgaggttcagacgtggctgcagcagatcggtttctccaagtactgcgagagcttccgggagcagcaggtggatggcgacctgcttctgcggctcacggaggaggaactccagaccgacctgggcatgaaatcgggcatcacccgcaagaggttctttagggagctcacggagctcaagaccttcgccaactattctacgtgcgaccgcagcaacctggcggactggctgggcagcctggacccgcgcttccgccagtacacctacggcctggtcagctgcggcctggaccgctccctgctgcaccgcgtgtctgagcagcagctgctggaagactgcggcatccacctgggcgtgcaccgcgcccgcatcctcacggcggccagagaaatgctacactccccgctgccctgtactggtggcaaacccagtggggacactccagatgtcttcatcagctaccgccggaactcaggttcccagctggccagtctcctgaaggtgcacctgcagctgcatggcttcagtgtcttcattgatgtggagaagctggaagcaggcaagttcgaggacaaactcatccagagtgtcatgggtgcccgcaactttgtgttggtgctatcacctggagcactggacaagtgcatgcaagaccatgactgcaaggattgggtgcataaggagattgtgactgctttaagctgcggcaagaacattgtgcccatcattgatggcttcgagtggcctgagccccaggtcctgcctgaggacatgcaggctgtgcttactttcaacggtatcaagtggtcccacgaataccaggaggccaccattgagaagatcatccgcttcctgcagggccgctcctcccgggactcatctgcaggctctgacaccagtttggagggtgctgcacccatgggtccaacctag

SEQ ID NO: 5 - DNA, 31 bp, Artificial Sequence
  PCR Primer
tctagagccaccatggtcctgacgctgcttc

SEQ ID NO: 6 - DNA, 26 bp, Artificial Sequence
  PCR Primer
gaattcttaggttggacccatgggtg